(19) United States
(12) Patent Application Publication
(10) Pub. No.: US 2003/0003142 A1
(43) Pub. Date: Jan. 2, 2003

(54) Composition and method for the treatment of pigmentation disorders

(76) Inventors: Mitchell S. Wortzman, Scottsdale, AZ (US); Eugene H. Gans, Westport, CT (US)

Correspondence Address: WILLIAM J. MCNICHOL, JR., ESQ., REED SMITH LLP, 2500 One Liberty Place, 1650 Market Street, Philadelphia, PA, 19103, US

(21) Appl. No.: 09/864,349

(22) Filed: May 23, 2001

(51) Int. Cl. A61K 31/07; A61K 31/05; A61K 9/127

(52) U.S. Cl. 424/450; 514/725; 514/734

(57) ABSTRACT

A superior skin depigmentation composition and a method for the depigmentation of skin is provided. The depigmenting composition contains effective amounts of hydroquinone and a retinoid.

### BACKGROUND OF THE INVENTION

This invention relates to compositions and methods for the treatment of pigmentation disorders in skin.

Pigmentation disorders in human skin are commonplace. Such disorders take a variety of forms. In many instances, pigmentation is uneven or mottled. In some instances, spots or patches where pigmentation is entirely lost may arise, a condition sometimes known as vitiligo. Blotches of dark pigmentation may occur during pregnancy a condition known as melasma. A similar condition may result from the use of oral contraceptives.

These disorders are often treated with depigmenting agents so as to reduce pigmentation when excessive amounts of pigment are present, or to reduce pigmentation around areas where pigment is absent. Several depigmenting agents are known. Among these are kojic acid, retinoids and hydroquinone. Hydroquinone is perhaps the best known of these, and its use dates back to the 1950's. The use of retinoids, in particular retinoic acid, retinal, and retinol is of more recent origin but has demonstrated some effectiveness.

All of these treatments are known to have shortcomings and drawbacks. As would be expected from a skin bleaching compound, all suffer from some degree of undesirable irritation to the skin, resulting in some degree of erythema, dryness, edema, burning, stinging, itching, or tightness of the skin. Use of depigmenting agents necessarily requires a balancing of effectiveness against the severity of these undesirable side effects. In one approach to this problem there have been developed especially effective carriers that allow depigmenting agents to be applied to the skin while minimizing undesirable side effects. This can allow for enhanced depigmentation, while reducing undesired side effects. See, for example, U.S. Pat. Nos. 6,057,630 and 5,932,612.

Another approach calls for the use of multiple depigmenting agents. For example, Swinyer, et al. reported the alternating use (mornings and evenings) of hydroquinone and retinoic acid in a depigmenting regimen. Swinyer, L. J. and Wortzman, M. S., “Study of Hydroquinone USP 4%, 0.05% Tretinoin, and in Combination in UV-induced Dyschromia with Actinic Photodamage,” Cosmetic Dermatology, 2000, pp. 13-18. This alternating, two-drug therapy produced no greater depigmenting effect than the use of hydroquinone alone.

### SUMMARY OF THE INVENTION

In accordance with this invention, a composition is provided that provides superior depigmenting action without excessive or increased skin irritation. It has been found that the simultaneous application of hydroquinone and a retinoid, especially retinol, results in enhanced depigmentation of the skin without a corresponding increase in skin irritation, and also results in a remarkable reduction of fine lines, wrinkles, and surface roughness of the skin.

### DETAILED DESCRIPTION OF THE INVENTION

Compositions according to the present invention contain hydroquinone. Hydroquinone may be present in any effective amount, typically from about 1% to about 12%, more typically from 1% to 8%. Non-prescription products typically contain 1% to 2%. Prescription products can contain higher concentrations. Some products contain concentrations 2% to 8%, more commonly, between 2% and 4%, with concentrations in the range of 3% to 4% most preferred.

Compositions according to the present invention also contain a retinoid, such as retinoic acid, retinal, retinol, their isomers, analogs and derivatives, such as isotrentoin, adapalene and tazarotene. Retinol is the most preferred retinoid. Retinoids may be used in any effective amount, typically within the range from about 0.01% to 5.0%, preferably from about 0.05% to 1%, and most preferably from about 0.025% to 0.5%. In some embodiments, from about 0.025% to about 2.0% is preferred.

It has also been found that compositions according to this invention may be advantageously compounded by providing the retinoid in a protective system. The protective system can be an entrapment system, a single or multi-laminar system, such as by the formation of vesicles such as a liposome or by utilizing wax, paraffin, silicone, polyethylene, or any material or system which protects the retinoid from oxidation. The preferred protective retinoid is in the form of small beads or vesicles which are of a form that can be adjusted to be incorporated into varied topical compositions. One skilled in the art is familiar with the known protective system technologies, such as encapsulation and entrapment methodologies. A preferred embodiment utilizes encapsulation. For use herein, the encapsulation forms a protective system to prohibit or inhibit the oxidation of the retinoid. An example of a protective system is Suncaps® formulated by Particle Sciences (a.k.a. Sun Smart) of Bethlehem, Pa.

In advantageous embodiments of the present invention, the pH of the composition is near neutrality, such as within a range of from 5.5 to 8.0, and preferably from 5.5 to 7.5. A pH of from 6.0 to 7.5 is most preferred. This may require the use of suitable buffering agents, as well as antioxidant stabilizers such as sulfites, including sulfites, bisulfites, metabisulfites, their salts and derivatives, including sodium metabisulfite.

Compositions according to this invention include a dermatologically acceptable carrier or vehicle. Such vehicles are widely known in the art and deliver the composition's ingredients to the skin without excessive degradation, inactivation or other unwanted interaction. An acceptable vehicle also possesses suitable aesthetic and cosmetic qualities, and may include emollients, conditioners and the like. Compositions according to this invention may include additives or components to enhance the skin penetration of its ingredients. They may also include ingredients with other therapeutic actions, such as antiinflamatories, antibiotics, exfoliants, and peels.

A composition according to the present invention was formulated as follows.

TABLE I

| Trade Name | CTFA Name | Percent |
|---|---|---|
| PHASE A | | |
| Purified Water | Purified Water | 45.25 |
| PHASE B | | |
| Carbomer 940 | Carbomer | 0.03 |
| Disodium EDTA | Disodium EDTA | 0.10 |
| PHASE C | | |
| Lecinol S-10 | Hydrogenated Lecithin | 0.75 |
| PHASE D | | |
| Phenyl Trimethicone | Phenyl Trimethicone | 4.00 |
| Gransil GCM-5 | Cyclopentasiloxane, | 2.50 |
| | Polysilicone-11 | |
| CK-100 | Dimethiconol | 0.39 |
| PHASE E | | |
| Linoleic Acid | Linoleic Acid | 2.50 |
| Cetyl alcohol | Cetyl alcohol | 2.75 |
| Lipomulse 165 | Glyceryl Stearate | 3.20 |
| | (and) PEG-100 | |
| | Stearate | |
| Cosmowax J | Cetearyl Alcohol | 1.50 |
| | (and) Ceteareth | |
| BHT | Butylated | 0.05 |
| | Hydroxytoluene | |
| Vitamin E Acetate | Tocopheryl Acetate | 0.75 |
| PHASE F | | |
| Sepigel 305 | Polyacrylamide (and) | 1.75 |
| | C 13-14 isoparaffin | |
| | (and) laureth-7 | |
| PHASE G | | |
| Purified Water | Purified Water | 2.00 |
| Sodium Metabisulfite | Sodium Metabisulfite | 0.25 |
| PHASE H | | |
| Purified Water | Purified Water | 3.00 |
| VC-PMGU | Magnesium L-Ascorbyl | 0.50 |
| | Phosphate | |
| PHASE I | | |
| Purified Water | Purified Water | 5.00 |
| Alcohol SDA 40, 200 | Alcohol | 3.00 |
| Proof | | |
| Glycerin 99% USP | Glycerin 99% USP | 4.00 |
| Hydroquinone | Hydroquinone | 4.00 |
| PHASE J | | |
| Purified Water | Purified Water | 1.00 |
| Triethanolamine 99% | Triethanolamine 99% | 0.60 |
| PHASE K | | |
| Benzyl alcohol | Benzyl Alcohol | 0.50 |
| Fragrance MAIDA J- | Fragrance | 0.03 |
| 9145 | | |
| Phenoxetol | Phenoxyethanol | 0.60 |
| PHASE L | | |
| Suncaps A-1283 | Water, Soybean | 10.00 |
| | (Glycine Soja) Oil, | |
| | Carnauba (Copernicia | |
| | Cerifera), wax, | |
| | tocopherol, retinol, | |
| | Ceteareth-20 | |

These phases are combined in a mixing tank with an in-line homogenizer as follows. Phase A is added to the tank, which is held at a temperature of from about 70° to 75° C., and in which a CO₂ atmosphere is maintained. Phase B is added, and mixing is initiated. Phase C is added and the homogenizer is engaged. Phase D is heated to from about 70° to 75° C. in a separate vessel with mixing until a clear, uniform phase is obtained. Phase D is then added to the main vessel. Phase E is heated to from about 70° to 75° C. in a separate vessel with mixing until a clear, uniform phase is obtained. Phase E is then added to the main vessel. Phase F is then added directly to the main vessel. The homogenizer is then disengaged and the vessel is cooled to about 50° C. Phase G is mixed in a separate vessel until clear, and then CO₂ is slowly introduced to the phase. Phase G is then added to the main vessel. Phase H is mixed in a separate vessel, and CO₂ is added to the phase before it is added to the main vessel. Phase I is then added directly to the main vessel. Phase J is premixed in a separate vessel with CO₂ and then added to the main vessel. The vessel is then cooled to about 25° to 27° C. Phase K is premixed in a separate vessel with CO₂, and then added to the main vessel. Phase L is premixed in a separate vessel and then added to the main vessel. The finished product is introduced into a container that has been flooded with Argon gas and then sealed.

While this composition is highly effective as a depigmenter in a variety of situations, its effectiveness is well demonstrated by its use to treat overall facial pigmentation. A composition according to this invention containing 4% hydroquinone, 0.3% retinol and having a pH of 6.9 was applied to the skin of patients having such conditions. The depigmentation results are summarized in Table II, along with the results of the application of a 4% hydroquinone preparation (SOLAQUIN FORTE, ICN Pharmaceuticals), a conventional retinoic acid preparation (RETIN-A, Ortho) and a conventional 3% hydroquinone preparation (MELANEX Neutrogena). In these tests, participants regularly applied the products to their pigmented lesions and their effects were evaluated visually by dermatologists on a scale of 1 to 5, using the MASI grading system.

TABLE II
PERCENT REDUCTION OF PIGMENTATION

| | Invention | Retin-A | Melanex | Solaquin Forte |
|---|---|---|---|---|
| Baseline | 0.0 | 0.0 | 0.0 | 0.0 |
| Week 4 | 18.5 | 4.4 | 1.11 | 5.66 |
| Week 8 | 34.8 | 17.4 | 1.11 | 9.43 |
| Week 12 | 45.8 | 25.0 | 8.52 | 5.66 |

From this data it can be seen that the combination of hydroquinone and a retinoid was more effective than either therapy alone. Furthermore, this enhanced depigmentation effect is not merely additive, as the percent reduction in pigmentation achieved by this invention is greater than that achieved by these two ingredients separately, thus demonstrating a synergistic depigmenting effect. This result is all the more surprising in light of the fact that retinol is believed to be less potent than retinoic acid.

Surprisingly, this enhanced depigmentation effect is achieved without a corresponding increase in skin irritation.

It has also been observed that the depigmentation effect obtained by use of this invention is also accompanied by a remarkable reduction in fine lines, wrinkles, and surface roughness of the skin, a surprising and beneficial effect.

This invention has been described with reference to specific embodiments, but is defined only by the following claims.

We claim:

1. A composition suitable for topical application to the skin, comprising effective amounts of hydroquinone, and a retinoid, all in a dermatologically acceptable carrier.

2. A composition according to claim 1, wherein the retinoid is selected from the group consisting of retinal, retinoic acid and retinol.

3. A composition according to claim 2, wherein the retinoid is retinol.

4. A composition according to claim 3, wherein the retinol is provided in a protective system.

5. A composition according to claim 4, wherein the protective system is an encapsulation.

6. A composition according to claim 4, wherein the protective system is a liposome.

7. A composition according to claim 4, wherein the protective system is a lamellar structure.

8. A composition according to claim 1, 2, 3, 4, 5, 6, or 7, wherein the retinoid is present in a concentration of from 0.01% to 5.00%.

9. A composition according to claim 8, wherein the retinoid is present in a concentration of from 0.05% to 1.0%.

10. A composition according to claim 8, wherein the retinoid is present in a concentration of from 0.025% to 0.50%.

11. A composition according to claim 8, wherein the pH of the composition is from 5.5 to 8.0.

12. A composition according to claim 8, wherein the pH of the composition is from 5.5 to 7.5.

13. A composition according to claim 8, wherein the pH of the composition is from 6.0 to 7.5.

14. A composition according to claim 1, 2, 3, 4, 5, 6, or 7, wherein the hydroquinone is present in a concentration of from 1% to 12%.

15. A composition according to claim 1, 2, 3, 4, 5, 6, or 7, wherein the hydroquinone is present in a concentration of from 2% to 8%.

16. A composition according to claim 1, 2, 3, 4, 5, 6, or 7, wherein the hydroquinone is present in a concentration of from 3% to 4%.

17. A composition according to claim 14, wherein the pH of the composition is from 5.5 to 8.0.

18. A composition according to claim 14, wherein the pH of the composition is from 5.5 to 7.5.

19. A composition according to claim 14, wherein the pH of the composition is from 6.0 to 7.5.

20. A method of treating disorders of skin pigmentation comprising the topical application of a composition containing effective amounts of hydroquinone, a retinoid, and a dermatologically acceptable carrier.

21. A method according to claim 20, wherein the retinoid is selected from the group consisting of retinal, retinoic acid and retinol.

22. A method according to claim 21, wherein the retinoid is retinol.

23. A method according to claim 22, wherein the retinol is provided in a protective system.

24. A method according to claim 23, wherein the protective system is an encapsulation.

25. A method according to claim 24, wherein the protective system is a liposome.

26. A method according to claim 25, wherein the protective system is a lamellar structure.

27. A method according to claim 1, 2, 3, 4, 5, 6, or 7, wherein the retinoid is present in a concentration of from 0.01% to 1.00%.

28. A method according to claim 8, wherein the retinoid is present in a concentration of from 0.05% to 1.0%.

29. A method according to claim 8, wherein the retinoid is present in a concentration of from 0.025% to 0.50%.

30. A method for the treatment of fine lines and wrinkles of skin, comprising the topical application of a composition containing effective amounts of hydroquinone, a retinoid, and a dermatologically acceptable carrier.

31. A method according to claim 30, wherein the retinoid is selected from the group consisting of retinal, retinoic acid and retinol.

32. A method according to claim 31, wherein the retinoid is retinol.

33. A method according to claim 32, wherein the retinol is provided in a protective system.

34. A method according to claim 33, wherein the protective system is an encapsulation.

35. A method according to claim 34, wherein the protective system is a liposome.

36. A method according to claim 35, wherein the protective system is a lamellar structure.

37. A method according to claim 1, 2, 3, 4, 5, 6, or 7, wherein the retinoid is present in a concentration of from 0.01% to 5.00%.

38. A method according to claim 8, wherein the retinoid is present in a concentration of from 0.05% to 1.0%.

39. A method according to claim 8, wherein the retinoid is present in a concentration of from 0.025% to 0.50%.
